(19) United States
(12) Patent Application Publication
(10) Pub. No.: US 2024/0055131 A1
(43) Pub. Date: Feb. 15, 2024

(54) MRTHOD, DEVICE FOR DISEASE PREDICTION, ELECTRONIC DEVICE AND COMPUTER-READABLE STORAGE MEDIUM

(71) Applicant: BOE Technology Group Co., Ltd., Beijing (CN)

(72) Inventor: Zhenzhong Zhang, Beijing (CN)

(73) Assignee: BOE Technology Group Co., Ltd., Beijing (CN)

(21) Appl. No.: 17/764,468

(22) PCT Filed: Apr. 29, 2021
(86) PCT No.: PCT/CN2021/090971

§ 371 Date: Mar. 28, 2022

(51) Int. Cl. G16H 50/30; G06N 3/045; G06N 3/084

(52) U.S. Cl. CPC G16H 50/30; G06N 3/045; G06N 3/084

(57) ABSTRACT

A method and a device for disease prediction, an electronic device and a computer-readable storage medium are provided. A method for predicting disease comprising the steps of: respectively acquiring a first feature and a second feature of a target object; inputting the first feature and the second feature into a risk prediction model, wherein the risk prediction model comprises a linear sub-model and a non-linear sub-model obtained by joint training; processing the first feature through the linear sub-model to obtain a first risk score; processing the second feature through the non-linear sub-model to obtain a second risk score; calculating a disease risk of the target subject based on the first risk score and the second risk score.

### TECHNICAL FIELD

The present disclosure relates to the field of computer technology, and in particularly, to a method and a device for disease prediction, an electronic device and a computer-readable storage medium.

### BACKGROUND

Disease prediction refers to a method for predicting the risk of an object suffering from a certain disease based on the relevant data of the object's physical state, living habits and so on. Disease prediction can predict the object's disease risk, so as to early prevent and make targeted treatment, and help to improve the diagnosis and treatment effect for the object.

### SUMMARY

Some embodiments of the present disclosure provide a method for predicting disease risk including the steps of:
respectively acquiring a first feature and a second feature of a target object;
inputting the first feature and the second feature into a risk prediction model, wherein the risk prediction model includes a linear sub-model and a non-linear sub-model;
processing the first feature through the linear sub-model to obtain a first risk score;
processing the second feature through the non-linear sub-model to obtain a second risk score;
calculating a disease risk of the target object based on the first risk score and the second risk score.

In some embodiments, the processing the first feature through the linear sub-model to obtain a first risk score includes:
processing the first feature through the linear sub-model to obtain a first risk score;
the linear sub-model is Formula 1;

Formula 1 is:

$$Pr ( Y = k \vert X = x ) = \frac{exp ( {β}_{k o} + {β}_{k}^{T} x )}{1 + {\sum }_{l}^{K - 1} exp ( {β}_{l0} + {β}_{l}^{T} x )} , k = 1 , 2 , 3 , 4 … K - 1 , l = 0 , 1 , 2 , 3 , K - 1 ;$$

$$Pr ( Y = k \vert X = x ) = \frac{1}{1 + {\sum }_{l}^{K - 1} exp ( {β}_{l0} + {β}_{l}^{T} x )} , k = K ;$$

X is an input variable, Y is an output variable, and the value range of the output variable is 1, 2, 3 . . . K; x is an input variable corresponding to the first feature; Pr(Y=k|X=x) is a probability of the first risk score being k when the input variable is x, βₖ is the kth model coefficient corresponding to the linear sub-model, and βₖ₀ is a scalarized value corresponding to the kth model coefficient.

In some embodiments, the non-linear sub-model includes a neural network model.

In some embodiments, the first feature includes an expert feature and the second characteristic includes a text feature.

In some embodiments, the disorder to be predicted is gestational hypertension, the expert characteristics including at least one of eating status, drinking status, smoking status, family history of coronary heart disease, family history of pregnancy-induced hypertension, mean arterial pressure, body mass index, birth weight, vaginal bleeding status, abortion record, preparation cycle; and/or

The text feature includes a medical record of the target object.

In some embodiments, prior to inputting the first feature and the second feature into a risk prediction model, the method includes:
obtaining a risk prediction model by joint training the linear sub-model and the non-linear sub-model;
the loss function 2 of the joint training is:

loss2=−Σᵢ₌₁^{N}yᵢ log Pr(yᵢ);
Pr(yi) shows the label from which the risk prediction model predicts the ith training data.

In some embodiments, prior to joint training the linear sub-model and the non-linear sub-model to obtain the risk prediction model, the method includes:
obtaining a model coefficient 3 of the linear sub-model by model training through Formula 2 and Formula 3;

Formula 2 is:

β=argmax_{β}L(β);

Formula 3 is:

L(β)=1/NΣᵢ^{N}Σₖ₌₁³I(yᵢ=k)log(Pr(yᵢ|xᵢ))

In some embodiments, prior to joint training the linear sub-model and the non-linear sub-model to obtain the risk prediction model, the method includes:
obtaining a non-linear sub-model by model training;
the loss function 1 of model training is:
the loss function 1 is:

loss1=−Σᵢ₌₁^{N}yᵢ log Pr2(yᵢ);

Pr2 (yᵢ) shows the label of the non-linear sub-model predicting the ith training data.

In some embodiments, the calculating a disease risk of the target object based on the first risk score and the second risk score, the method includes:
calculating the disease risk of the target subject through Formula 4;

Formula 4 is:

Pr=λ×Pr1(Y)+(1−λ)×Pr2(Y)

Pr is the disease risk of the target object, Pr1 (Y) is a first risk score, Pr2 (Y) is a second risk score, k is a preset proportionality factor, k is less than or equal to 1 and greater than or equal to 0.

Some embodiments of the present disclosure provide a disease prediction device including:
a feature acquisition configured to respectively acquire a first feature and a second feature of the target object;
an input module configured to input the first feature and the second feature into a risk prediction model, wherein the risk prediction model includes a linear sub-model and a non-linear sub-model obtained by joint training;
a first risk score determination configured to process the first feature through the linear sub-model to obtain a first risk score;
a second risk score determination configured to process the second feature through the non-linear sub-model to obtain a second risk score;
a disease risk calculation configured to calculate a disease risk of the target object based on the first risk score and the second risk score.

Some embodiments of the present disclosure provide an electronic device including a processor, a memory, and a computer program stored on the memory and executable on the processor, which when executed by the processor, performs the steps of the disease prediction method according to some aspects of the present disclosure.

Some embodiments of the present disclosure provide a computer readable storage medium having stored thereon a computer program which, when executed by a processor, performs the steps of the disease prediction method of some aspects of the present disclosure.

### BRIEF DESCRIPTION OF THE DRAWINGS

In order to more clearly illustrate the technical solutions of the embodiments of the present disclosure, a brief description will be given below with reference to the accompanying drawings which are required to be used in the description of the embodiments of the present disclosure; it is obvious that the drawings in the following description are only some embodiments of the present disclosure, and it would have been obvious for a person of ordinary skill in the art to obtain other drawings according to these drawings without involving any inventive effort.

FIG. 1 is a flow diagram of a disease prediction method provided by some embodiments of the present disclosure;

FIG. 2 is a workflow diagram of a non-linear sub-model according to some embodiments of the present disclosure;

FIG. 3 is yet another flow diagram of a disease prediction method provided by some embodiments of the present disclosure; and

FIG. 4 is a structure diagram of a disease prediction device provided by some embodiments of the present disclosure.

### DETAILED DESCRIPTION

Reference will now be made in detail to the embodiments of the present disclosure, examples of which are illustrated in the accompanying drawings. It is to be understood that the present disclosure is intended to be illustrative of the embodiments of the present disclosure, and is not intended to be exhaustive of the embodiments of the present disclosure. Based on the embodiments in the present disclosure, all other embodiments obtained by a person of ordinary skill in the art without inventive effort fall within the scope of the present disclosure.

Some embodiments of the present disclosure provide a disease prediction method, and an execution object of the method may be any electronic device, for example, may be applied to an application program having a disease prediction function, and the method may be executed by a server or a terminal device of the application program, and optionally, the method may be executed by the server.

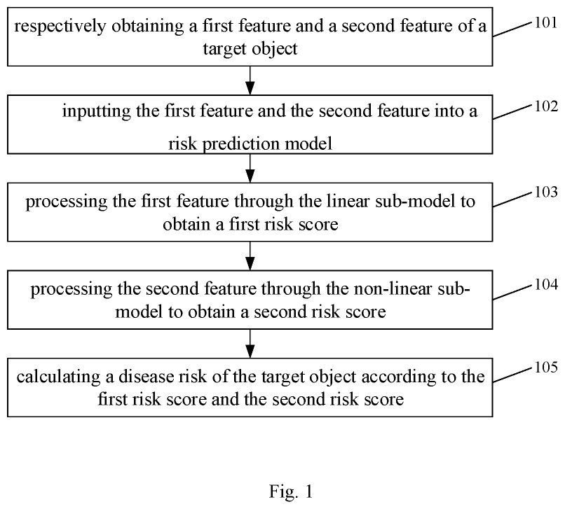

As shown in FIG. 1, in some embodiments, the disease prediction method includes the steps of:

Step 101: respectively obtaining a first feature and a second feature of a target object.

The target object refers to an object requiring disease risk prediction, for example, any registered user of the above-mentioned application. The first feature and the second feature refer to information about the target object provided according to certain requirements or criteria respectively.

The first feature and the second feature contain information about the target object, which information can be processed to obtain a disease risk of the target object.

In some embodiments, the first feature and the second feature may be received by a terminal device, and may also be obtained by a database, wherein the database may be constructed from user information obtained by performing a questionnaire survey on the user, or may also be constructed from user information obtained by analyzing historical behavior data of the user.

In some embodiments, the first feature includes an expert feature, wherein the expert feature is designed by professionals such as doctors and has a significant influence factor on the risk of the disease to be predicted, and the expert feature may be set by the professionals according to the factors related to the risk of the disease to be predicted, such as the living habit, the history of genetic diseases and the physical state of the target object.

It should be understood that an object's disease risk may be influenced by a variety of intrinsic and extrinsic factors, including genetic factors that may contribute to the disease risk, while extrinsic factors, including living environment and habits, may contribute to the subject's disease risk. Meanwhile, the physical state of the target object may fluctuate over a period of time, for example, when the living environment and living habits of the target subject have not changed, the target object may also have fluctuating physical states such as an occasional cold, which may affect the user's disease risk.

In some embodiments, the disorder to be predicted is gestational hypertension, and the expert features include one or more of eating status, drinking status, smoking status, family history of coronary heart disease, family history of pregnancy-induced hypertension, mean arterial pressure, BMI index (Body Mass Index), birth weight, vaginal bleeding status, miscarriage record, preparation cycle.

Obviously, when predicting the disease risk of different conditions, the factors included in the set first feature can be adjusted adaptively, and specifically, professionals such as professional doctors can set a corresponding problem according to the influencing factors for the disease so as to collect the relevant first feature of the target object.

The link between expert features and disease risk can be effectively captured by designing the required acquired expert characteristics based on professional knowledge by professionals.

It will be appreciated that if too few are designed for the first feature, the prediction of disease risk may be less accurate, however, manually designing the first feature requires expertise and if too many designs are made for the first feature, more resources are expended.

Illustratively, when predicting the risk of a disease, 10 expert features are designed by professionals, and 80% of the important information about the risk of the disease is already known. However, if 90% of the important information about the risk of the disease needs to be known, 100 expert features may need to be designed. For the professionals who design expert features, the contents covered by the new expert features are more specific. For the design of each expert feature, the effort is greater relative to the original expert features. For the target object, there are too many items to be filled or validated, however, these additional first features do contribute relatively little to the risk of disease, namely, the input resources do not match the predicted contribution to the risk of disease.

Based on the above analysis, it can be seen that in practical applications, there is a certain limit on the design number of expert features, and due to the limit on the number of expert features, a part of information may not be covered, for example, 20% of information is missed by the above 10 expert features.

In some embodiments, a second feature is introduced, in some embodiments, the second feature is a text feature, and in some embodiments, the second feature specifically includes a medical record of the target object.

In some embodiments, a medical record is a qualified medical record recorded at the request or direction of professionals, such as professional doctors, which may include more comprehensive information about the object.

Step 102: inputting the first feature and the second feature into a risk prediction model, where the risk prediction model includes a linear sub-model and a non-linear sub-model.

After the first feature and the second feature are determined, the first feature and the second feature are input into a risk prediction model. In some embodiments, the risk prediction model includes a Wide & Deep model.

The linear sub-model is a Wide model, wherein the Wide model can include a Logistic Regression (LR) model, and the LR uses a linear function to model the posterior probability of a class mark, and can directly output a normalized probability of an interval from 0 to 1, which helps to reduce the complexity of calculation, and the processing effect of the first feature which has been set is better.

The nonlinear sub-model is a deep model, which has faster learning speed and higher learning accuracy, and helps to improve the processing speed.

Step 103: processing the first feature through the linear sub-model to obtain a first risk score.

In some embodiments, the linear sub-model determines a first risk score by analyzing a first feature.

Illustratively, the diet status may include the question of whether the target Object uses fruit heavily 15 weeks prior to pregnancy, which can be unambiguously determined as “yes” or “no” depending on the amount of fruit used by the pregnant woman as compared to certain criteria. For another example, for the existence of family history of coronary heart disease and family history of pregnancy-induced hypertension, these two questions can be definitely answered “Existence” or “Absence”, and for the questions of mean arterial pressure, BMI index, birth weight, vaginal bleeding status, miscarriage record, preparation cycle, etc. all can be definitely numerical.

Since the expert features are designed, and definite results can be given for these expert features, such as the above-mentioned positive response or negative response, and the above-mentioned time-dependent expert features include a determined time value, and the results corresponding to these expert features have a direct impact on the risk of disease; therefore, these expert features can be processed through a linear sub-model to obtain a corresponding first risk score.

Specifically, the response to the set question is input as a first feature into a linear sub-model in the risk prediction model, and a corresponding first risk score can be obtained.

The processing the first feature through the linear sub-model to obtain a first risk score, including:
substituting the first feature into Formula 1 to obtain a first risk score;
wherein Formula 1 is:

$$Pr ( Y = k \vert X = x ) = \frac{exp ( {β}_{k o} + {β}_{k}^{T} x )}{1 + {\sum }_{l}^{K - 1} exp ( {β}_{l0} + {β}_{l}^{T} x )} , k = 1 , 2 , 3 , 4 … K - 1 , l = 0 , 1 , 2 , 3 , K - 1$$

$$Pr ( Y = k \vert X = x ) = \frac{1}{1 + {\sum }_{l}^{K - 1} exp ( {β}_{l0} + {β}_{l}^{T} x )} , k = K$$

wherein X is an input variable, Y is an output variable, and the value range of the value y of the output variable is 1, 2, 3 . . . K; x is an observation value of the input variable corresponding to the first feature; Pr(Y=k|X=x) is a probability of the first risk score being k when the input variable is x; and β is a model coefficient corresponding to the linear sub-model, and more specifically, βₖ is a kth model coefficient corresponding to the linear sub-model, and βₖ0 is a scalarized value corresponding to the kth model coefficient.

In some embodiments, x is a P-dimensional vector, wherein each dimension corresponds to an expert feature, illustratively, wherein each dimension can be one of the above-mentioned eating status, drinking status, smoking status, family history of coronary heart disease, family history of pregnancy-induced hypertension, mean arterial pressure, body mass index, birth weight, vaginal bleeding status, abortion record, pregnancy preparation period, etc. In some embodiments, the dimension of x is four dimensions if four features of family history of pregnancy induced hypertension, mean arterial pressure, body mass index, and birth weight are set, and the dimension of x is 11 dimensions if eleven features of diet, alcohol consumption, smoking, family history of coronary heart disease, family history of pregnancy induced hypertension, mean arterial pressure, body mass index, birth weight, vaginal bleeding status, abortion record, and preparation period are set. The observation value of the input variable refers to the result corresponding to the above-mentioned first feature. The expert feature set for the above-mentioned smoking status is specifically “whether there is smoking status in the first 15 weeks of pregnancy”. The corresponding observation value may include the results of “absence” and “presence”, and may also include “absence”, “presence, smoking amount less than 5 cigarettes per day” and “presence, smoking amount greater than 5 cigarettes per day”.

The observed value of the first risk score refers to the specific result of the first risk score. For example, there may be a greater risk, a certain risk and no risk, and the observation value may be specifically replaced with a corresponding numerical value; for example, it may be 0, 1, 2, or −1, 0, 1, etc. Illustratively, the first risk score may also be represented by a numerical value between 0 and 1, representing a corresponding probability value. Obviously, the manner of representation is not limited thereto.

It should be understood that the LR model can be represented by the following representation formula:

$$log \frac{Pr ( Y = 1 \vert X = x )}{Pr ( Y = K \vert X = x )} = {β}_{10} + {β}_{1}^{T} x ;$$

$$log \frac{Pr ( Y = 2 \vert X = x )}{Pr ( Y = K \vert X = x )} = {β}_{20} + {β}_{2}^{T} x ;$$

$$…$$

$$log \frac{Pr ( Y = K - 1 \vert X = x )}{Pr ( Y = K \vert X = x )} = {β}_{( K - 1 ) 0} + {β}_{K - 1}^{T} x ;$$

$${\sum }^{K} Pr ( Y = k \vert X = x ) = 1 .$$

The representation formula of the LR model is derived to obtain the above Formula 1.

Step 104: processing the second feature through the non-linear sub-model to obtain a second risk score.

In some embodiments, the second feature includes a medical record of the target object. The nonlinear sub-model may include at least one of an RNN (Recurrent Neural Network) model and a GRU (Gated Recurrent Neural Networks) model.

In some embodiments, the non-linear sub-model includes an LSTM (Long Short-Term Memory) model. Lstm model has long-term memory function and is easy to implement, which can help to reduce the system load and the difficulty of modeling, and improve the accuracy of feature extraction.

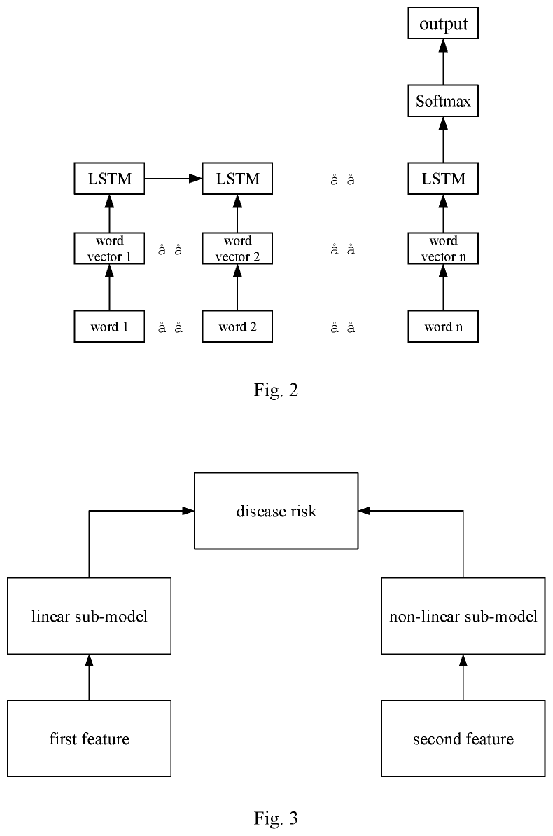

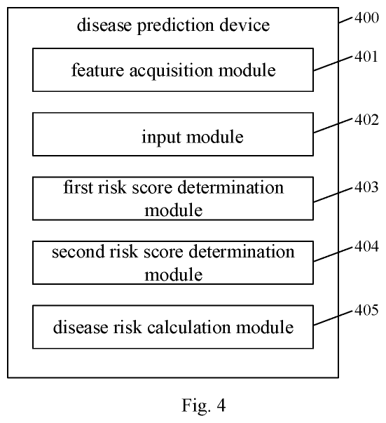

As shown in FIG. 2, the input may be all words included in the medical record, where word i represents the ith word in the medical record, i=1, 2, 3 . . . n. Converting the text into a word vector, inputting same into a non-linear sub-model, and after classifying the output result of the non-linear sub-model, obtaining the output result as a second risk score.

In one embodiment, the second risk score is calculated by the formula:

Pr2(Y)=softmax(W×h_{T})

wherein Pr2 (Y) is a second risk score, softmax ( ) represents a softmax function and hT represents an hidden state at the last moment of a word vector, and since the corresponding hidden state of each word is determined by the word vector corresponding to the word and the hidden state corresponding to the previous word, the implicit state at the last moment actually includes all the information about the input text, so that information omission can be avoided.

In some embodiments, the word vector is a 512 dimensional vector, and the text vector includes useful information and some other information, and when the useful information therein is extracted, the amount of data is reduced, and therefore, the useful information therein can be represented by a low-dimensional vector, so as to save storage space and improve data processing speed.

Illustratively, the hidden state dimension is illustrated as 256 dimensions. Obviously, the actual dimensions are not limited thereto and can be set according to actual needs.

W is an N*M matrix, wherein M is equal to the dimension of the hidden state, and when the dimension of the hidden state is 256, M is also equal to 256; N is the number of labels that output the result, for example, when using the softmax function for classification, three results can be obtained: there are no target diseases, there may be target diseases, and there are target diseases; these three results respectively correspond to three labels of 1 to 3; accordingly, the value of N is equal to 3; obviously, when the number of set results changes, the number of labels also changes, and accordingly, the value of N also changes.

Thus, by inputting the medical record of the target subject, the second risk score Pr2 (Y) can be obtained. The output result Pr2 (Y) includes the probabilities that the values of Y are 1, 2 and 3, respectively, and therefore the obtained second risk score Pr2 (Y) is an N*1-dimensional vector, here specifically a 3*1-dimensional vector.

Step 105: calculating a disease risk of the target object according to the first risk score and the second risk score.

After determining the first risk score and the second risk score, combining the first risk score and the second risk score can obtain a comprehensive score for the disease risk of the target object, and the calculated disease risk can relatively accurately reflect the disease risk of the target object.

In some embodiments, the calculating a disease risk for the target object according to the first risk score and the second risk score includes:
calculating the disease risk of the target subject by the following Equation 4;

pr=λ×Pr1(Y)+(1-λ)×Pr2(Y) Formula 4;

Pr is the disease risk of the target object, Pr1 (Y) is a first risk score, Pr2 (Y) is a second risk score, λ is a preset proportionality factor, k is less than or equal to 1 and greater than or equal to 0.

The λ may be understood as a weighting factor that adjusts the ratio of the first risk score and the second risk score, illustratively, taking 0.8, then Pr=0.8Pr1 (Y)+0.2Pr2 (Y). Obviously, when actually needed, a corresponding preset proportion coefficient λ can be set according to the actual situation, so as to improve the accuracy of the prediction on the risk of diseases.

The disease risk of the target object may be provided to the user, for example, may be pushed to the user through a floating window, station letter, etc. When the user logs into the corresponding APP. In addition, when a change in the user feature is detected, for example, when the user modifies the input feature, the user may be pushed again in the form of a short message or other prompt information, for example, the user may also be recommended by means of voice broadcast, etc.

As shown in FIG. 3, by using a linear sub-module to determine a first risk score according to a set first feature, and by using a non-linear sub-module to determine a second risk score according to a second feature, and combining the first risk score and the second risk score to obtain a disease risk of the target object, it is helpful to improve the accuracy of the disease risk assessment.

In some embodiments, the inputting the first and second features into a risk prediction model further includes a step of model training. In this embodiment, the linear sub-model and the non-linear sub-model are independently trained first, and after the independent training of the linear sub-model and the non-linear sub-model is completed, the linear sub-model and the non-linear sub-model are further jointly trained.

In some embodiments, the step of training the linear sub-model includes:

The model coefficients 3 of the linear sub-model are obtained by model training through Formula 2 and Formula 3.

Formula 2 is:

β=argmax_{β}L(β);
Formula 3 is:

L(β)=1/NΣᵢ^{N}Σₖ₌₁³I(yᵢ=k)log(Pr(yᵢ|xᵢ))

In some embodiments, the step of training the non-linear sub-model includes:
obtaining a non-linear sub-model by model training with loss function 1;
obtaining a non-linear sub-model by model training;
the loss function 1 of model training is:
the loss function 1 is:

loss1=−Σᵢ₌₁^{N}yᵢ log Pr2(yᵢ);

In some embodiments, after training the linear sub-model and the non-linear sub-model is completed, further including: the risk prediction model is obtained by jointly training the linear sub-model and the nonlinear sub-model;
the loss function 2 of the joint training is:

loss2=−Σᵢ₌₁^{N}yᵢ log Pr(yᵢ);

In model training, firstly, given training data {(x1, y1), (x2, y2), . . . , (xN, yN)}, wherein xi represents a first feature in the training data, and yi has values of 1, 2 and 3 respectively, which respectively represent not having the target disease, possibly having the target disease and having the target disease, and can be obtained by professionals through manual annotation. Obviously, the above-mentioned target diseases refer to diseases requiring risk prediction, and may be exemplified by diseases such as gestational hypertension.

In the implementation, a linear sub-model is first obtained by model training, specifically, the above Formula 2 is obtained by a maximum likelihood method, and the model parameter 3 of the linear sub-model is determined by a Quasi-Newton descent method.

In the above-mentioned formula, argmax 0 represents a argmax function, I represents an indication function. When yᵢ is equal to k, the value of I (yi=k) is 1, otherwise it is 0. represents the probability that the label is yᵢ when the input feature is xi.

Next, model training is performed on the non-linear sub-model according to the above loss function 1, for example, learning parameters by minimizing the loss function through a stochastic gradient descent method, and when a certain training condition is satisfied (for example, when the loss function converges or a certain number of iterations is satisfied, etc.), the nonlinear sub-model satisfying the usage requirement is obtained. Wherein, in the loss function loss 1, Pr2 (yi) represents the label probability of the non-linear sub-model predicting the ith training data.

Illustratively, in some embodiments, when i is equal to 1, yᵢ has a value of 1, representing no target disease, and the probability of having no target disease is 0.5, then Pr2 (yi)=0.5.

Finally, the linear sub-model and the non-linear sub-model are jointly trained by a loss function 3, for example, learning parameters by minimizing the loss function by a stochastic gradient descent method, and when certain training conditions are satisfied (for example, when the loss function converges or a certain number of iterations are satisfied, etc.), a risk prediction model satisfying the use requirements is obtained. In the loss function loss 2, Pr (yi) represents the label probability that the risk prediction model predicts the ith training data.

Some embodiments of the present disclosure provide a disease prediction device.

In some embodiments, the disease prediction device 400 includes:
a feature acquisition module 401 for respectively acquiring a first feature and a second feature of a target object;
an input module 402 configured to input the first feature and the second feature into a risk prediction model, wherein the risk prediction model includes a linear sub-model and a non-linear sub-model obtained by joint training;
a first risk score determination module 403 for processing the first feature through the linear sub-model to obtain a first risk score;
a second risk score determination module 404 for processing the second feature through the non-linear sub-model to obtain a second risk score;
a disease risk calculation module 405 for calculating a disease risk of the target subject according to the first risk score and the second risk score.

In some embodiments, the first risk score determination module 403 is specifically configured to: processing the first feature through the linear sub-model to obtain a first risk score;
the linear sub-model is Formula 1;

Formula 1 is:

$$Pr ( Y = k \vert X = x ) = \frac{exp ( {β}_{k o} + {β}_{k}^{T} x )}{1 + {\sum }_{l}^{K - 1} exp ( {β}_{l0} + {β}_{l}^{T} x )} , k = 1 , 2 , 3 , 4 … K - 1 , l = 0 , 1 , 2 , 3 , K - 1 ;$$

$$Pr ( Y = k \vert X = x ) = \frac{1}{1 + {\sum }_{l}^{K - 1} exp ( {β}_{l0} + {β}_{l}^{T} x )} , k = K ;$$

X is an input variable, Y is an output variable, x is an observation value of the input variable corresponding to the first feature, and the value range of the output variable is 1, 2, 3 . . . K; Pr(Y=k|X=x) is a probability of the first risk score being k when the input variable is x, which is the kth model coefficient corresponding to the linear sub-model, and which is a scalarized value corresponding to the kth model coefficient.

In some embodiments, the non-linear sub-model includes a neural network model.

In some embodiments, the first feature includes an expert feature and the second feature includes a text feature.

In some embodiments, the disorder to be predicted is gestational hypertension, the expert features including at least one of eating status, drinking status, smoking status, family history of coronary heart disease, family history of pregnancy-induced hypertension, mean arterial pressure, body mass index, birth weight, vaginal bleeding status, abortion record, preparation cycle; and/or

The text feature includes a medical record of the target object.

In some embodiments, further including:
a joint training module for jointly training the linear sub-model and the non-linear sub-model to obtain a risk prediction model;
the loss function 2 of the joint training is:

loss2=−Σᵢ₌₁^{N}yᵢ log Pr(yᵢ);
Pr(yᵢ) represents the label probability that the risk prediction model predicts the ith training data.

In some embodiments, further including:
a first training module for obtaining a model coefficient R of the linear sub-model by performing model training via formulae 2 and 3;
Formula 2 is:

β=argmax_{β}L(β);
Formula 3 is:

L(β)=1/NΣᵢ^{N}Σₖ₌₁³I(yᵢ=k)log(Pr(yᵢ|xᵢ))

In some embodiments, further including:
a second training module for obtaining a non-linear sub-model through model training;
the loss function 1 of model training is:

loss1=−Σᵢ₌₁^{N}yᵢ log Pr2(yᵢ);

Pr2(yi) represents the label probability of the non-linear sub-model predicting the ith training data.

In some embodiments, the disease risk calculation module 405 is specifically configured to calculate a disease risk of the target object by Formula 4;

Formula 4 is:

Pr=λ×Pr1(Y)+(1−λ)×Pr2(Y);

Pr is the disease risk of the target object, Pr1 (Y) is a first risk score, Pr2 (Y) is a second risk score, λ is a preset proportionality factor, k is less than or equal to 1 and greater than or equal to 0.

Embodiments of the present disclosure also provide an electronic device including a processor, a memory, and a computer program stored in the memory and operable on the processor, which when executed by the processor, implements the respective processes of the above-mentioned embodiments of the disease prediction method and achieves the same technical effects, and will not be described in detail herein.

The embodiments of the present disclosure also provide a computer-readable storage medium having stored thereon a computer program which, when executed by a processor, implements the respective processes of the above-mentioned embodiments of the disease prediction method and can achieve the same technical effects, and will not be described in detail herein. Wherein the computer readable storage medium, such as a Read-Only Memory (ROM), a Random Access Memory (RAM), a magnetic disk or an optical disk, etc.

Those of ordinary skill in the art would recognize that the various illustrative modules, units, and algorithm steps described in connection with the embodiments disclosed herein can be implemented as electronic hardware, or computer software, or combinations of computer software and electronic hardware. Whether such functionality is implemented as hardware or software depends upon the particular application and design constraints imposed on the technical solution. Those skilled in the art may implement the described functionality in varying ways for each particular application, but such implementation decisions should not be interpreted as causing a departure from the scope of the present disclosure.

It will be clear to those skilled in the art that, for convenience and brevity of description, reference may be made to the corresponding processes in the above-described method embodiments for the specific workings of the systems, devices and units described above and will not be described in detail herein.

It should be understood that, although the various steps in the flow diagrams of the drawings are shown in order as indicated by the arrows, the steps are not necessarily performed in the order indicated by the arrows. The steps are performed in no strict order unless explicitly stated herein, and may be performed in other orders. Furthermore, at least some of the steps in the flow diagrams of the drawings may include sub-steps or stages, which are not necessarily performed at the same time, but may be performed at different times, in a different order, or in a different order, and may be performed in turn or in alternation with at least some of the other steps or sub-steps or stages of other steps.

In the examples provided herein, it should be understood that the disclosed device and methods may be implemented in other ways. For example, the device embodiments described above are merely illustrative, for example the division of elements is merely a logical function division, and in practice there may be additional divisions, for example elements or components may be combined or integrated into another system, or some features may be omitted, or not performed. In another aspect, the couplings or direct couplings or communication connections shown or discussed with respect to each other may be indirect couplings or communication connections through some interface, means, or element, and may be electrical, mechanical, or otherwise.

The units described as separate elements may or may not be physically separated, and the elements shown as units may or may not be physical units, namely. May be located in one place, or may be distributed over a plurality of network units. Some or all of the units may be chosen to achieve the objectives of embodiments of the present disclosure according to actual needs.

In addition, various functional units in various embodiments of the present disclosure may be integrated in one processing unit, may exist physically in a single unit, or may be integrated in one unit with two or more units.

The functions, if implemented in the form of software functional units and sold or used as stand-alone products, may be stored in a computer-readable storage medium. Based on such an understanding, the technical solutions of the present disclosure, either inherently or in any part contributing to the prior art, may be embodied in the form of a software product stored in a storage medium, comprising instructions that enable a computer device, which may be a personal computer, a server, or a network device, to perform all or part of the steps of the methods described in the various embodiments of the disclosure. Whereas the above-mentioned storage medium comprises: various media can store the program code, such as U-disk, removable hard disk, ROM, RAM, magnetic or optical disk.

The above is only specific embodiments the present disclosure, but the application range of the present disclosure is not limited thereto. Any person skilled in the art who can easily think of changes or substitutions within the technical scope disclosed in the disclosure shall be covered by the protection scope of the present disclosure. Therefore, the protection scope of the present disclosure shall be subject to the protection scope defined by the claims.

1. A method for predicting disease risk, comprising:
respectively acquiring a first feature and a second feature of a target object;
inputting the first feature and the second feature into a risk prediction model, wherein the risk prediction model comprises a linear sub-model and a non-linear sub-model;
processing the first feature through the linear sub-model to obtain a first risk score;
processing the second feature through the non-linear sub-model to obtain a second risk score; and
calculating a disease risk of the target object according to the first risk score and the second risk score.

2. The method of claim 1, wherein the processing the first feature through the linear sub-model to obtain a first risk score comprises:
processing the first feature through the linear sub-model to obtain a first risk score;
the linear sub-model is Formula 1;
wherein Formula 1 is:
$$Pr ( Y = k \vert X = x ) = \frac{exp ( {β}_{k o} + {β}_{k}^{T} x )}{1 + {\sum }_{l}^{K - 1} exp ( {β}_{l0} + {β}_{l}^{T} x )} , k = 1 , 2 , 3 , 4 … K - 1 , l = 0 , 1 , 2 , 3 , K - 1 ;$$
$$Pr ( Y = k \vert X = x ) = \frac{1}{1 + {\sum }_{l}^{K - 1} exp ( {β}_{l0} + {β}_{l}^{T} x )} , k = K$$
wherein X is an input variable, Y is an output variable, and a value range of the output variable is 1, 2, 3 . . . K; x is an input variable corresponding to the first feature;
Pr(Y=k|X=x) is a probability of the first risk score being k when the input variable is x, βₖ is the kth model coefficient corresponding to the linear sub-model, and βₖ₀ is a scalarized value corresponding to the kth model coefficient.

3. The method of claim 1, wherein the non-linear sub-model comprises a neural network model.

4. The method of claim 1, wherein the first feature comprises an expert feature and the second feature comprises a text feature.

5. The method of claim 4, wherein the disease risk to be predicted is gestational hypertension and the expert feature comprises at least one of eating status, drinking status, smoking status, family history of coronary heart disease, family history of pregnancy-induced hypertension, mean arterial pressure, body mass index, birth weight, vaginal bleeding status, abortion record, preparation cycle; and/or
the text feature comprises a medical record of the target object.

6. The method of claim 1, wherein prior to inputting the first and second features into a risk prediction model, the method further comprises:
obtaining a risk prediction model by joint training the linear sub-model and the non-linear sub-model;
wherein a loss function of the joint training is:
loss2=−Σᵢ₌₁^{N}yᵢ log Pr(yᵢ);
wherein Pr(yi) represents a label probability that the risk prediction model predicts an i-th training data.

7. The method of claim 6, wherein prior to jointly training the linear sub-model and the non-linear sub-model to obtain the risk prediction model, the method further comprises:
obtaining a model coefficient β of the linear sub-model by performing model training through Formula 2 and Formula 3;
wherein Formula 2 is:
β=argmax_{β}L(β);
Formula 3 is:
$$L ( β ) = \frac{1}{N} {\sum }_{i}^{N} {\sum }_{k = 1}^{3} I ( {y}_{i} = k ) log ( Pr ( {y}_{i} \vert {x}_{i} ) ) .$$

8. The method of claim 6, wherein prior to jointly training the linear sub-model and the non-linear sub-model to obtain the risk prediction model, the method further comprises:
obtaining a non-linear sub-model by model training;
wherein a loss function of model training is:
loss1=−Σᵢ₌₁^{N}yᵢ log Pr2(yᵢ);
wherein Pr2(yi) represents a label probability of the non-linear sub-model predicting the i-th training data.

9. The method of claim 1, wherein the calculating a disease risk of the target object based on the first risk score and the second risk score comprises:
calculating the disease risk of the target object through Formula 4;
wherein Formula 4 is:
Pr=λ×Pr1(Y)+(1-λ)×Pr2(Y);
Pr is the disease risk of the target object, Pr1 (Y) is a first risk score, Pr2 (Y) is a second risk score, λ is a preset proportionality factor, λ is less than or equal to 1 and greater than or equal to 0.

10. (canceled)

11. An electronic device comprising a processor, a memory and a computer program stored on the memory and executable on the processor, when the computer program is executed by the processor to perform:
respectively acquiring a first feature and a second feature of a target object;
inputting the first feature and the second feature into a risk prediction model, wherein the risk prediction model comprises a linear sub-model and a non-linear sub-model;
processing the first feature through the linear sub-model to obtain a first risk score;
processing the second feature through the non-linear sub-model to obtain a second risk score; and
calculating a disease risk of the target object according to the first risk score and the second risk score.

12. (canceled)

13. The electronic device of claim 11, wherein the computer program is executed by the processor to perform:
processing the first feature through the linear sub-model to obtain a first risk score;
the linear sub-model is Formula 1;
wherein Formula 1 is:
$$Pr ( Y = k \vert X = x ) = \frac{exp ( {β}_{k o} + {β}_{k}^{T} x )}{1 + {\sum }_{l}^{K - 1} exp ( {β}_{l0} + {β}_{l}^{T} x )} , k = 1 , 2 , 3 , 4 … K - 1 , l = 0 , 1 , 2 , 3 , K - 1 ;$$
$$Pr ( Y = k \vert X = x ) = \frac{1}{1 + {\sum }_{l}^{K - 1} exp ( {β}_{l0} + {β}_{l}^{T} x )} , k = K$$
wherein X is an input variable, Y is an output variable, and a value range of the output variable is 1, 2, 3 . . . K; x is an input variable corresponding to the first feature; Pr(Y=k|X=x) is a probability of the first risk score being k when the input variable is x, βₖ is the kth model coefficient corresponding to the linear sub-model, and βₖ₀ is a scalarized value corresponding to the kth model coefficient.

14. The electronic device of claim 11, wherein the non-linear sub-model comprises a neural network model.

15. The electronic device of claim 11, wherein the first feature comprises an expert feature and the second feature comprises a text feature.

16. The electronic device of claim 15, wherein the disease risk to be predicted is gestational hypertension and the expert feature comprises at least one of eating status, drinking status, smoking status, family history of coronary heart disease, family history of pregnancy-induced hypertension, mean arterial pressure, body mass index, birth weight, vaginal bleeding status, abortion record, preparation cycle; and/or
the text feature comprises a medical record of the target object.

17. The electronic device of claim 11, wherein prior to inputting the first and second features into a risk prediction model, the computer program is executed by the processor to perform:
obtaining a risk prediction model by joint training the linear sub-model and the non-linear sub-model;
wherein a loss function of the joint training is:
loss2=−Σᵢ₌₁^{N}yᵢ log Pr(yᵢ);
wherein Pr(yi) represents a label probability that the risk prediction model predicts an i-th training data.

18. The electronic device of claim 17, wherein prior to jointly training the linear sub-model and the non-linear sub-model to obtain the risk prediction model, the computer program is executed by the processor to perform:
obtaining a model coefficient β of the linear sub-model by performing model training through Formula 2 and Formula 3;
wherein Formula 2 is:
β=argmax_{β}L(β);
Formula 3 is:
$$L ( β ) = \frac{1}{N} {\sum }_{i}^{N} {\sum }_{k = 1}^{3} I ( {y}_{i} = k ) log ( Pr ( {y}_{i} \vert {x}_{i} ) ) .$$

19. The electronic device of claim 17, wherein prior to jointly training the linear sub-model and the non-linear sub-model to obtain the risk prediction model, the computer program is executed by the processor to perform:
obtaining a non-linear sub-model by model training;
wherein a loss function of model training is:
loss1=−Σᵢ₌₁^{N}yᵢ log Pr2(yᵢ);
wherein Pr2(yi) represents a label probability of the non-linear sub-model predicting the i-th training data.

20. The electronic device of claim 11, wherein the computer program is executed by the processor to perform:
calculating the disease risk of the target object through Formula 4;
wherein Formula 4 is:
Pr=λ×Pr1(Y)+(1-λ)×Pr2(Y);
Pr is the disease risk of the target object, Pr1 (Y) is a first risk score, Pr2 (Y) is a second risk score, λ is a preset proportionality factor, λ is less than or equal to 1 and greater than or equal to 0.
